(19) United States
(12) Patent Application Publication
(10) Pub. No.: US 2023/0233751 A1
(43) Pub. Date: Jul. 27, 2023

(54) NASAL IRRIGATION DEVICE

(71) Applicant: Luisam Tarrats, San Juan, PR (US)

(72) Inventor: Luisam Tarrats, San Juan, PR (US)

(21) Appl. No.: 18/099,893

(22) Filed: Jan. 20, 2023

Related U.S. Application Data
(60) Provisional application No. 63/302,031, filed on Jan. 22, 2022.

(51) Int. Cl. A61M 3/02

(52) U.S. Cl. CPC A61M 3/0279; A61M 2210/0618

(57) ABSTRACT

Disclosed are nasal irrigation devices having an adjustable nozzle. Various designs for the nasal irrigation devices are provided.

### CROSS REFERENCE TO RELATED APPLICATIONS

This patent application claims the benefit of U.S. Provisional Patent Application 63/302,031 filed 22 Jan. 2022; which is incorporated herein by reference for all purposes.

### TECHNICAL FIELD

The present invention in general is related to systems and methods for washing body cavity. More particularly, the present invention is related to an apparatus for discharging a fluid to the nasal cavity of a user.

### BACKGROUND

Nasal cleansing involves washing out nasal and sinus passages with a suitable solution. The solution is made to circulate in and out of the sinus cavities, coming back out from the mouth or from the opposite side of the user's nose. This is believed to be helpful in maintaining nasal hygiene, limiting sinus infection and in relieving symptoms and signs of rhinitis. Also, in pre and post-operative periods, nasal irrigation can be used to flush out bacteria, clots, and normal crusts.

Nasal irrigation is now widely recognized as a treatment for chronic rhinosinusitis and during the postoperative period. Topical sinus irrigations play a critical role in the management of sinonasal disease, and the improvement in irrigant penetration into the sinuses postoperatively greatly contributes to the success of endoscopic sinus surgery. High-volume irrigation delivery is more effective for achieving distribution to the sinuses that other topical delivery methods such as nasal sprays, nebulizers or atomizers. The improvement of sinus irrigation penetration thus increasingly becomes an important outcome of endoscopic sinus surgery.

Powered nasal irrigation systems that may include a suctioning option are not generally available for home use. Devices such as squeeze bottle, gravity feed neti pots, collapsible deformable bulbs etc., are commercially available for home use. Also, available for home use are devices that use a motorized pump or other electromechanical means to provide a fluid flow for nasal irrigation. Many of these devices require the user's head to be tilted to one side during use. This is not always convenient or comfortable for the user. For users with certain medical conditions, leaning of the head to irrigate the nasal passage may not be possible at all. The problem may get aggravated if the flow and pressure of the fluid are not controlled and splashing or overflow of the fluid occurs. Also, the conventional bottles or containers used for nasal irrigation are not ergonomically convenient for the users. Variability related to the overall shape of the nose, presence of cartilaginous components and skin thickness makes use of conventional nasal irrigation devices even more challenging.

Accordingly, systems and methods that overcome the above-mentioned disadvantages associated with conventional nasal irrigation devices, such as providing better sinus penetration and coverage of the nasal cavity, and that account for different nasal configurations, are desirable and an object of the present disclosure.

### SUMMARY

The following presents a simplified summary in order to provide a basic understanding of some aspects of the present disclosure. This summary is not an extensive overview, and it is not intended to identify key/critical elements or to delineate the scope thereof. Its sole purpose is to present some concepts in a simplified form as a prelude to the more detailed description that is presented later.

The present disclosure is directed to a nasal irrigation device having an adjustable nozzle. In certain embodiments the nasal irrigation device of the present disclosure comprises a container having a storage space for a nasal rinsing solution, a dispenser and an adapter. The container having a large opening is capable of holding adequate quantity of the nasal rinsing solution and also facilitates mixing of the solution. The adapter on one side surrounds the mouth of the container and holds the dispenser on the other side. A ball-and-socket kind of coupling between a socket disposed on the adapter and a substantially spherical mounting end of the dispenser enables rotation of the dispenser with respect to a longitudinal axis of the container in an assembled condition. The socket has a tube extension which defines a fluid passageway between the container and the dispenser. The coupling allows flow of fluid through the fluid passageway from the container to the nozzle end at any inclined position of the dispenser within an allowable range of angle of inclination. An angle limiter projecting inwardly to the socket from the top end of the tube extension prevents the dispenser from being tilted to a degree wherein the mounting end may block the fluid passageway.

A user may keep his/her head in a vertical position, put the nozzle tip in the nostril and tilt the container to hold it at any desired angle. A gentle squeeze of the resilient sidewall of the container propels the nasal rinsing solution through the nozzle end to the nasal passage of the user.

The present disclosure also provides a nasal irrigation device comprising: a) a container comprising a top end, a bottom end, a bottom surface, an upper portion and a lower portion, the top end defining a neck portion comprising an inner surface and an outer surface; b) an inner cap comprising an inner surface, an outer surface, a top end and a bottom end, the inner cap comprising a sealing ring proximal to the top end of the inner cap; c) a nozzle comprising an opening, a top portion, a middle portion and a bottom portion, the nozzle defining a passageway therethrough, the top portion of the nozzle defining a tip and the middle portion of the nozzle defining a ball joint, the nozzle engaging with the sealing ring of the inner cap; and d) a tube having a top end and a bottom end, the top end of the tube engaging with the bottom portion of the nozzle and the bottom end of the tube extending within the container from the top end of the container toward the bottom end of the container; wherein the angle of the nozzle is adjustable relative to the container when the nasal irrigation device is assembled.

In certain embodiments the outer surface of the neck portion comprises a first fitting and the inner surface of the inner cap comprises a second fitting, wherein the first fitting and the second fitting are designed to releasably engage with each other. In some embodiments the first fitting and the second fitting are threadably engaged when the nasal irrigation device is assembled. In other embodiments the first fitting and the second fitting form a snap fit connection when the nasal irrigation device is assembled. In further embodiments the inner surface of the neck portion comprises a third fitting and the outer surface of the inner cap comprises a fourth fitting, wherein the third fitting and the fourth fitting are designed to releasably engage with each other. In yet further embodiments the third fitting and the fourth fitting are threadably engaged when the nasal irrigation device is assembled. In still further embodiments the third fitting and the fourth fitting form a snap fit connection when the nasal irrigation device is assembled.

In some embodiments the nozzle is adjustable relative to the container when the nasal irrigation device is assembled based on a rotational position of the ball joint relative to the sealing ring of the inner cap. In various embodiments the opening has a stellate shape. In additional embodiments the nasal irrigation device further comprises an outer cap comprising a top end, a bottom end, an outer surface and an inner surface, the inner surface proximal to the bottom end of said outer cap designed to engage with the outer surface of the inner cap. In yet other embodiments the nasal irrigation device further comprises a time management strip attached to the bottom surface of the container.

In particular embodiments the container is comprised of a flexible polymer. In some embodiments the polymer comprises zinc or silver. In other embodiments the polymer comprises low-density polyethylene (LDPE). In further embodiments the tip of the nozzle is designed to fit within a nostril of a patient. In certain embodiments the tip of the nozzle has a semicircular shape. In additional embodiments the tip is comprised of rubber or a thermoplastic elastomer. In yet other embodiments the tip is comprised of a material that reduces the risk of at least one of nasal bleeding or septum perforation when the tip is inserted into a nostril. In some embodiments the tip is in the shape of a reverse taper. In particular embodiments the reverse taper shape provides for improved nasal fitting at the nostril level. In certain embodiments the middle portion of the nozzle is comprised of plastic. In some embodiments the middle portion of the nozzle comprises acrylonitrile butadiene styrene (ABS).

In additional embodiments the container further comprises a liquid, and wherein the nozzle is capable of varying the amount of the liquid passing through the nozzle. In some embodiments the liquid is a saline solution. In certain embodiments the container further comprises a flexible sidewall. In other embodiments the container further comprises a liquid, and wherein the nozzle is configured to create a turbulent stream of liquid exiting the nozzle when the sidewall is squeezed. In yet other embodiments the container further comprises a liquid, and wherein the nozzle is configured to create a level of turbulence in a stream of liquid exiting the nozzle when the sidewall is squeezed, such that the turbulent stream of liquid is directed toward and sufficient to enter and flush at least a substantial portion of a sinus passage.

In further embodiments the tip of the nozzle defines a stellate shaped opening. In yet further embodiments the tip of the nozzle defines an opening that increases surface area dispersion and intranasal turbulence. In additional embodiments the surface area dispersion and intranasal turbulence are sufficient to generate a flooding mechanism for irrigation of a sinus cavity. In some embodiments the upper portion of the container is angled relative to the lower portion of the container. In other embodiments the inner cap comprises ABS. In yet other embodiments the sealing ring comprises a thermoplastic elastomer.

In certain embodiments wherein upon engagement of the ball joint with the sealing ring the nozzle comprises multidirectional positioning capabilities. In some embodiments the nozzle is positioned to irrigate in at least one of a superior or inferior arrangement.

It is, therefore, an object of the present disclosure to provide an apparatus that enables adjustment of the nozzle in different angles. Another object of the present disclosure is to provide an ergonomically enhanced nasal irrigation device. Yet another object of the present disclosure is to provide an efficient nasal irrigation device. Another object of the present disclosure is to provide a nasal irrigation device that enables easy introduction of solution to nasal passage of a user. Yet another object of the present disclosure is to provide a device for effective irrigation of solution to all the desired parts of the nasal passage. Another object of the present disclosure is to provide a cost effective method for enabling a user to get his/her nasal passage irrigated while keeping his/her head in an upright position.

### BRIEF DESCRIPTION OF THE DRAWINGS

All of the figures depict preferred embodiments, although other embodiments are contemplated, and the present disclosure is not limited to the embodiments shown.

FIG. 1 is an outer appearance perspective view illustrating a nasal irrigation device in accordance with an embodiment of the present invention.

FIG. 2A illustrates a longitudinal sectional view of the nasal irrigation device in accordance with an embodiment of the present invention.

FIG. 2B is an enlarged view of the portion of the nasal irrigation device as illustrated in FIG. 2A and identified as detail 2B in FIG. 2A.

FIG. 3 is an exploded view of the nasal irrigation device in accordance with an embodiment of the present invention.

FIG. 4A illustrates the nasal irrigation device with the nozzle in tilted position in accordance with an embodiment of the present invention.

FIG. 4B illustrates longitudinal sectional view of the nasal irrigation device with the nozzle in tilted position in accordance with an embodiment of the present invention.

FIG. 5 illustrates fluid path in the nasal irrigation device in accordance with an embodiment of the present invention.

FIG. 6 illustrates the nasal irrigation device in use in accordance with an embodiment of the present invention.

FIG. 7 is an exploded view of the components of a nasal irrigation device according to certain embodiments of the present disclosure.

FIG. 8A is an illustration of a nasal irrigation device according to certain embodiments of the present disclosure. FIG. 8B is a perspective view of the nasal irrigation device shown in FIG. 8A. FIG. 8C is a front perspective view of the nasal irrigation device shown in FIG. 8A. FIG. 8D is a back perspective view of the nasal irrigation device shown in FIG. 8A. FIG. 8E is a right perspective view of the nasal irrigation device shown in FIG. 8A. FIG. 8F is a left perspective view of the nasal irrigation device shown in FIG. 8A. FIG. 8G is a top perspective view of the nasal irrigation device shown in FIG. 8A. FIG. 8H is a bottom perspective view of the nasal irrigation device shown in FIG. 8A.

FIG. 9A is an illustration of a nasal irrigation device with an outer cap according to certain embodiments of the present disclosure. FIG. 9B is a perspective view of the nasal irrigation device with an outer cap shown in FIG. 9A. FIG. 9C is a front perspective view of the nasal irrigation device with an outer cap shown in FIG. 9A. FIG. 9D is a back perspective view of the nasal irrigation device with an outer cap shown in FIG. 9A. FIG. 9E is a right perspective view of the nasal irrigation device with an outer cap shown in FIG. 9A. FIG. 9F is a left perspective view of the nasal irrigation device with an outer cap shown in FIG. 9A. FIG. 9G is a top perspective view of the nasal irrigation device with an outer cap shown in FIG. 9A. FIG. 9H is a bottom perspective view of the nasal irrigation device with an outer cap shown in FIG. 9A.

FIG. 10 is a cross-sectional view of a nasal irrigation device with an outer cap according to certain embodiments of the present disclosure.

FIG. 11A is an illustration of a container of a nasal irrigation device according to certain embodiments of the present disclosure. FIG. 11B is a perspective view of the container of a nasal irrigation device shown in FIG. 11A. FIG. 11C is a front perspective view of the container of a nasal irrigation device shown in FIG. 11A. FIG. 11D is a back perspective view of the container of a nasal irrigation device shown in FIG. 11A. FIG. 11E is a right perspective view of the container of a nasal irrigation device shown in FIG. 11A. FIG. 11F is a left perspective view of the container of a nasal irrigation device shown in FIG. 11A. FIG. 11G is a top perspective view of the container of a nasal irrigation device shown in FIG. 11A. FIG. 11H is a bottom perspective view of the container of a nasal irrigation device shown in FIG. 11A.

FIG. 12A is an illustration of a nozzle and inner cap of a nasal irrigation device according to certain embodiments of the present disclosure. FIG. 12B is an illustration of a nozzle, inner cap and tube of a nasal irrigation device according to certain embodiments of the present disclosure. FIG. 12C is a perspective view of the nozzle and inner cap of a nasal irrigation device shown in FIG. 12A. FIG. 12D is a front perspective view of the nozzle and inner cap of a nasal irrigation device shown in FIG. 12A. FIG. 12E is a back perspective view of the nozzle and inner cap of a nasal irrigation device shown in FIG. 12A. FIG. 12F is a right perspective view of the nozzle and inner cap of a nasal irrigation device shown in FIG. 12A. FIG. 12G is a left perspective view of the nozzle and inner cap of a nasal irrigation device shown in FIG. 12A. FIG. 12H is a top perspective view of the nozzle and inner cap of a nasal irrigation device shown in FIG. 12A. FIG. 12I is a bottom perspective view of the nozzle and inner cap of a nasal irrigation device shown in FIG. 12A. FIG. 12J is a perspective view of the nozzle, inner cap and tube of a nasal irrigation device shown in FIG. 12B.

FIG. 13A, FIG. 13B, FIG. 13C, FIG. 13D, FIG. 133, FIG. 14F and FIG. 13G are illustrations of time management strip time indicator labels according to certain embodiments of the present disclosure.

### DETAILED DESCRIPTION

To provide an overall understanding of the disclosure, certain illustrative embodiments and examples will now be described. However, it will be understood by one of ordinary skill in the art that the same or equivalent functions and sequences may be accomplished by different embodiments that are also intended to be encompassed within the spirit and scope of the disclosure. The compositions, apparatuses, systems and/or methods described herein may be adapted and modified as is appropriate for the application being addressed and that those described herein may be employed in other suitable applications, and that such other additions and modifications will not depart from the scope hereof.

In the following detailed description, numerous specific details are set forth in order to provide a thorough understanding of the present disclosure. However, it will be understood by those skilled in the art that the present disclosure may be practiced without these specific details. In other instances, well-known methods, procedures and components have not been described in detail so as not to obscure the present disclosure.

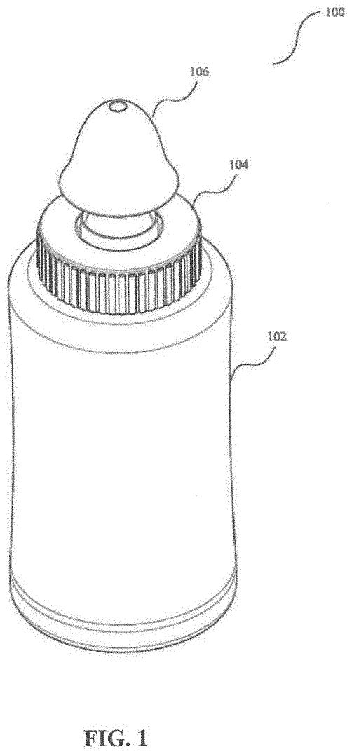

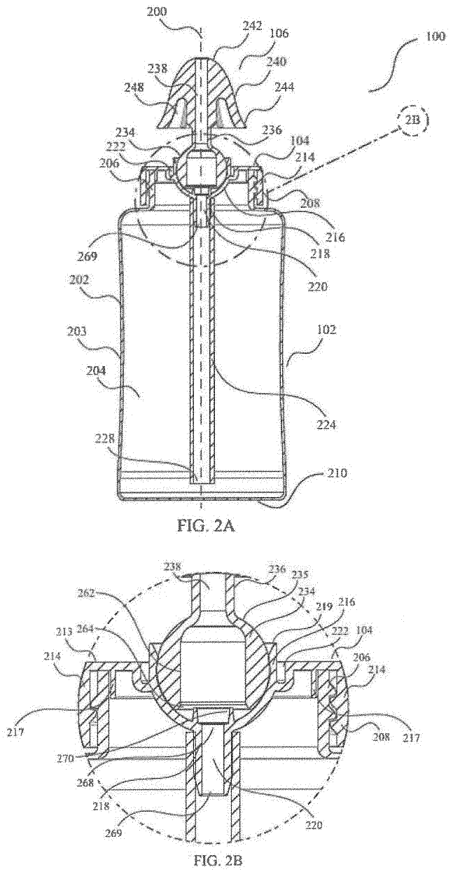

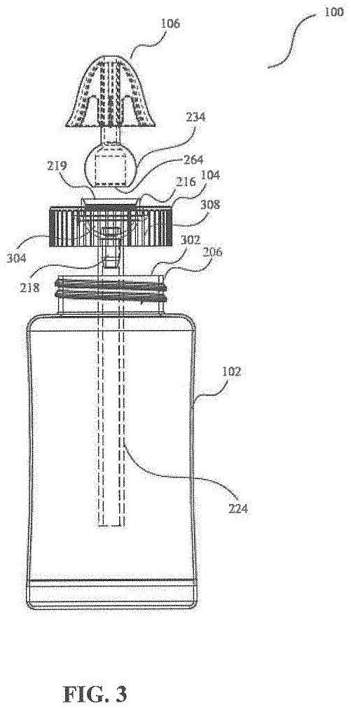

Reference to FIG. 1, the nasal irrigation device with adjustable nozzle 100, hereinafter referred to as nasal irrigation device 100, of one embodiment of the present disclosure comprises a container 102, an adapter 104 and a dispenser 106. As illustrated in FIG. 2A, FIG. 2B and FIG. 3, container 102 has a storage space 204 defined by a bottom end 210, a substantially cylindrical sidewall 202 extending perpendicularly from the periphery of the bottom end 210, and a mouth 206 transitioning from the sidewall 202 disposed distally opposite to the bottom end 210. The mouth 206 defines an opening 302 that gives access to the storage space 204 from outside. External threads are disposed on the outer periphery of the mouth 206. The storage space 204 can hold a nasal rinsing solution, i.e., a liquid. The container 102, particularly the sidewall 202, is made to resiliently deform under pressure. The container 102 can be dimensioned to be cylindrical, bulbous or any other suitable form. In a preferred embodiment, the sidewall 202 is given a concave configuration around a middle section 203 wherein the sidewall projects inwardly toward the longitudinal axis 200 at least to some extent resulting in a hyperboloid longitudinal section of the container 102.

The concave shape of the sidewall 202 provides a comfortable gripping location and contour for a user to hold the container 102. The resilient, flexible nature of the bottle material makes it convenient for the user to squeeze the container 102, particularly in the narrowed waist middle section 203, toward the longitudinal axis 200 to dispense a liquid stored in the storage space 204. The container 102 quickly rebounds to its original, non-squeezed shape immediately upon release of the squeeze pressure.

The adapter 104 is configured to connect the dispenser 106 rotatably or pivotally to the container 102 while allowing fluid communication between the two. As shown in FIG. 1, FIG. 2A, FIG. 2B and FIG. 3, the adapter 104 removably attaches the dispenser 106 to the container 102. It comprises top wall 213 and an annular wall 214 extending downwardly from the periphery of the top wall 213. The annular wall 214 defines a collar 208 that is dimensioned and shaped to surround and engage with the outer periphery of the mouth 206 to secure the adapter 104 to the container 102 while the top wall 213 rotatably and pivotally holds the dispenser 106 facing away from the mouth 206 but in fluid communication with the storage space 204. Inside of the collar 208 may define internal threads for engagement with the mouth 206. The collar 208 may comprise an inner collar 217 that is configured to fit inside the mouth 206 in an abutting relationship with the inner periphery of the mouth 206 to firmly support the top wall 213. The top wall 213 defines a socket 216 having a socket cavity 304 (indicated in FIG. 3) with a socket opening defined by a free open end 219, and a tube extension 218 opening into the socket cavity 304. In a preferred embodiment, the socket cavity 304 is substantially spherical in shape and the free open end 219 faces away from the mouth 206 when the adapter 104 is attached to the mouth 206. The free open end 219 extends along the longitudinal axis 200 at least above the great circle or the equatorial circle of the spherical cavity so that it can pivotally retain the mounting end 234 of the dispenser 106. The top wall 213 further defines a void space 222 around the free open end 219 to make the free open end 219 resilient so that it flexes open to receive the mounting end 234 and then provide a tight grip around it. In some embodiments, a plurality of vertically disposed ribs 308 (indicated in FIG. 3) are provided on the outer surface of the collar 208.

A top end 268 of the tube extension 218 opens into the socket cavity 304 and a distally opposite tube bottom end 269 of it opens into the storage space 204 when the adapter 104 is attached to the mouth 206 of the container 102 thereby forming a fluid passageway 220 between the storage space 204 and the socket cavity 304. An angle limiter 270 is disposed over the top end 268 of the tube extension 218 that projects inwardly into the spherical socket cavity 304. In some embodiments, an elongated tube 224 is affixed to the tube extension 218 so that the distal end 228 of the elongated tube 224 can reach the bottom of the storage space 204.

With continuing reference to FIG. 1, FIG. 2A, FIG. 2B and FIG. 3, the dispenser 106 includes an outer wall 235 and an inner wall 262. The outer wall 235 defines a nozzle end 240, the mounting end 234 and a neck region 236 interposed between the nozzle end 240 and the mounting end 234. Outer diameter of the neck region 236 is preferably made substantially less than the diameter of the great circle or the equatorial circle of the mounting end 234 so that the mounting end 234 can have more degrees of freedom to pivot while mounted within the socket cavity 304. The inner wall 262 defines a nozzle channel 238 extending from the nozzle end 240 to the mounting end 234. In a preferred embodiment, the nozzle channel 238 has an area of cross-section greater inside the mounting end 234 than in the rest of the nozzle channel length.

As illustrated in FIG. 2A, the nozzle end 240 comprises a nozzle tip 242 and a nozzle base 244. The nozzle end 240 is anatomically dimensioned to correspond to the entry region of a nasal passageway of a human being. Starting from the nozzle tip 242, the outer wall 235 gradually extends radially outward from the nozzle channel 238 and downward toward the nozzle base 244 to form a free skirt which facilitates the nozzle end 240 to conform to the nasal passageway of a user. In a preferred embodiment, a void underside 248 is defined inside and around the free skirt to provide flexibility.

The mounting end 234 has a spherical region defined by the outer wall 235 that is dimensioned to conform to at least a portion of the inner wall of the socket cavity 304 so that adequate sealing can be provided between the mounting end 234 and the socket cavity 304 against any leakage of fluid even when the mounting end 234 rotates within the socket cavity 304. The mounting end 234 has a truncated bottom face 264 disposed substantially perpendicular to the axis of the nozzle channel 238. As illustrated in FIG. 2B, the truncated bottom face 264 makes the mounting end 234 look like a spherical cap or spherical dome with the neck region 236 extending from the opposite side of the truncated bottom face 264 of the mounting end 234. The nozzle channel 238 opens at the truncated bottom face 264 and the inner wall 262 meets the outer wall 235 at the periphery of the truncated bottom face 264. In a preferred embodiment, the opening of the nozzle channel 238 at the truncated bottom face 264 extends to the periphery of the spherical portion of the outer wall 235.

The angle limiter 270 restricts the angle by which the dispenser 106 can incline/pivot with respect to the longitudinal axis 200 so that the spherical region of the mounting end 234 never blocks the top end 268 of the tube extension 218 when the dispenser 106 is rotated within the socket cavity 304. The area of cross-section of the nozzle channel 238 inside the mounting end 234 is dimensioned to be greater than an area of cross-section of the top end 268 of the tube extension 218 such that the angle limiter 270 is surrounded by at least a portion of the nozzle channel 238 in an assembled condition of the device 100.

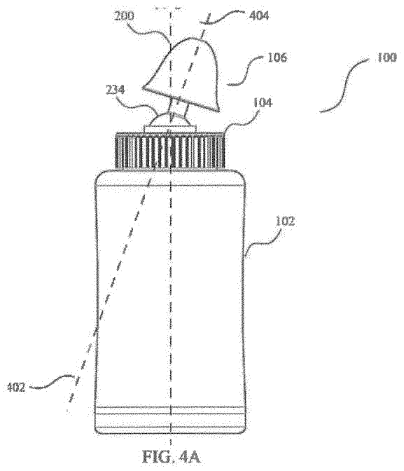

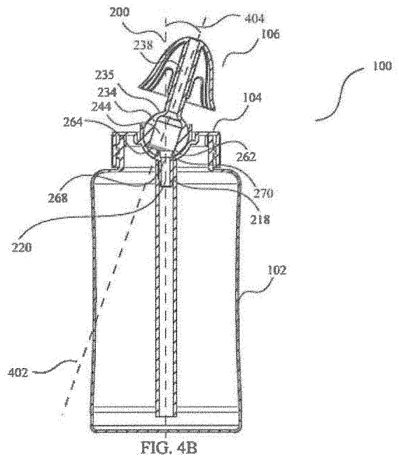

FIG. 4A and FIG. 4B show the dispenser 106 in a tilted position with the longitudinal axis 402 of the dispenser 106 making an angle of inclination 404 with the longitudinal axis 200 of the container 102. As can be seen in FIG. 4B, the angle limiter 270 comes in contact with the mounting end 234, preferably with the periphery of the nozzle channel 238 at the truncated bottom face 264, when the dispenser 106 is tilted to this allowable angle of inclination 404. That way, the angle limiter 270 prevents the outer wall 235 at the mounting end 234 from blocking the top end 268 of the tube extension 218 and, thus, preventing interruption in flow of fluid through the fluid passageway 220.

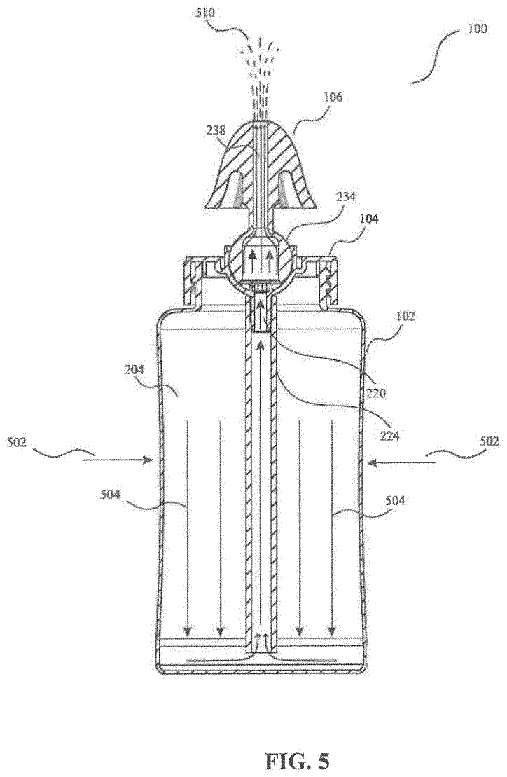

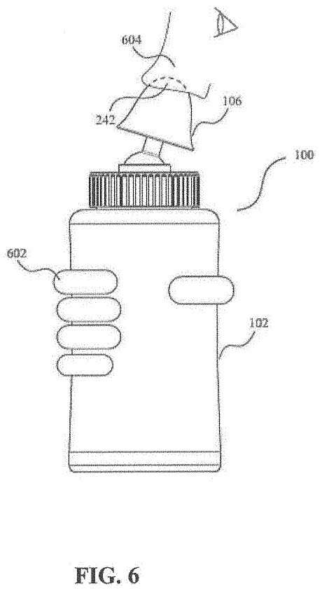

FIG. 5 depicts the path that is followed by the fluid, i.e., by the nasal rinsing solution stored in the storage space 204 when deformation of the sidewall 202 of the container 102 occurs due to application of pressure 502. The pressure 502 can be applied by squeezing the container 102 by a user's hand 602 as shown in FIG. 6. Difference between the atmospheric pressure outside the dispenser 106 and the inside pressure 504 in the storage space 204 causes the fluid to enter the elongated tube 224, pass through the fluid passageway 220 and exit the dispenser 106 through the nozzle channel 238 as a fluid spray/stream 510.

For use, the storage space 204 of the container 102 is filled with a nasal rinsing solution after unscrewing the adapter 104 from the mouth 206 of the container 102. A large opening 302 of the mouth 206 facilitates pouring of the solution to the container 102. The adapter 104 holding the dispenser 106 is then screwed back onto the mouth 206. As the dispenser 106 is rotatable within a wide range of allowable angles of inclination, the user is not required to tilt his/her head to get his/her nasal passage irrigated. Instead, the user can keep his/her head substantially vertical and tilt the dispenser 106 at a desired angle so that the nozzle tip 242 sealingly makes contact with the entrance of the nasal passage 604. The user may hold the container vertically or tilt it to any angle while maintaining the user's head at a vertical position and the nozzle tip 242 still engaged with the entrance of the nasal passage 604. To inject the solution to the nasal passage 604, the user may apply pressure and squeeze the container 102 with his/her hand 602. The pressure difference thus created will force the solution out of the container 102 through the dispenser 106 into the nasal passage 604. As the mounting end 234 of the dispenser 106 and the socket cavity 304 form an airtight/watertight seal, no leakage of fluid occurs even when the dispenser 106 or the container 102 is rotated/pivoted with respect to each other. Since, the angle of the dispenser 106 is changed as required, better irrigation can be achieved in all the sections of the nasal cavity including the frontal sinuses.

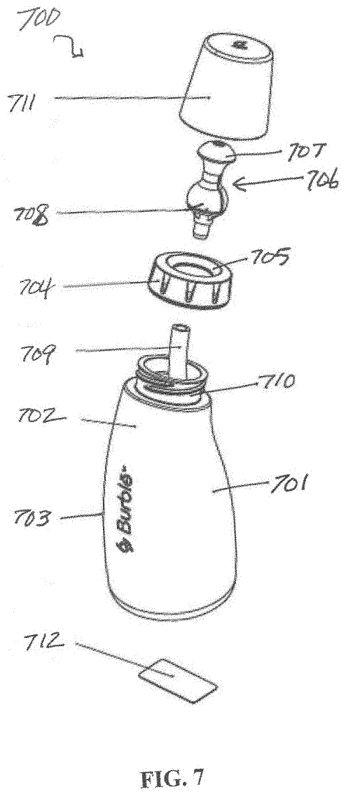

FIG. 7 shows an exploded view of another embodiment of the present disclosure. Nasal irrigation device 700, comprises a container 701 having a flexible sidewall 703, and a neck portion 702 comprising a first fitting 710, an inner cap 704 having a sealing ring 705, a nozzle 706 having a tip 707 and a ball joint 708, a tube 709, an outer cap 711 and a time management strip 712. The tip 707 is anatomically dimensioned to correspond to the entry region of a nasal passageway of a human being. The first fitting comprise external threads disposed on the outer periphery of the neck portion 702 of the container 701. Inside of the inner cap 704 may define internal threads for releasable engagement with the first fitting 710. Optionally the inner surface of the neck portion 702 can comprise a third fitting and the outer surface of the inner cap 705 can comprise a fourth fitting, wherein the third fitting and the fourth fitting are designed to releasably engage with each other. The container 701 can hold a nasal rinsing solution, i.e., a liquid. The container 701, particularly the flexible sidewall 703, is made to resiliently deform under pressure. The resilient, flexible nature of the container material makes it convenient for the user to squeeze the container 701 to dispense a liquid stored therein. The container 701 quickly rebounds to its original, non-squeezed shape immediately upon release of the squeeze pressure. The container 701 can be dimensioned to be cylindrical, bulbous or any other suitable form.

The sealing ring 705 is dimensioned to conform to at least a portion of the nozzle 706 so that adequate sealing can be provided between the inner cap 704 and the container 701 against any leakage of fluid even when the nozzle 706 rotates within the inner cap 704. The tube 709 is configured to connect the nozzle 706 rotatably or pivotally to the container 701 while allowing fluid communication between the two. The tube 709 removably attaches the nozzle 706 to the container 701. In some embodiments the tube 709 can reach the bottom of the container 701.

For use, the container 701 is filled with a nasal rinsing solution after unscrewing the inner cap 704 from the container 701. A large opening of the container 701 facilitates pouring of the solution to the container 701. The inner cap 704 holding the nozzle 706 is then screwed back onto the container 701. As the nasal irrigation device 700 is rotatable within a wide range of allowable angles of inclination, the user is not required to tilt his/her head to get his/her nasal passage irrigated. Instead, the user can keep his/her head substantially vertical and tilt the nasal irrigation device 700 at a desired angle so that the nozzle tip 707 sealingly makes contact with the entrance of the nasal passage of the user. The user may hold the container vertically or tilt it to any angle while maintaining the user's head at a vertical position and the nozzle tip 707 still engaged with the entrance of the nasal passage. To inject the solution to the nasal passage, the user may apply pressure and squeeze the container 701 with his/her hand. The pressure difference thus created will force the solution out of the container 701 through the nozzle 706 into the nasal passage. As the inner cap 704 and the container 701 form an airtight/watertight seal, no leakage of fluid occurs even when the nozzle 706 or the container 701 is rotated/pivoted with respect to each other. Since, the angle of the nozzle 706 is changed as required, better irrigation can be achieved in all the sections of the nasal cavity including the frontal sinuses.

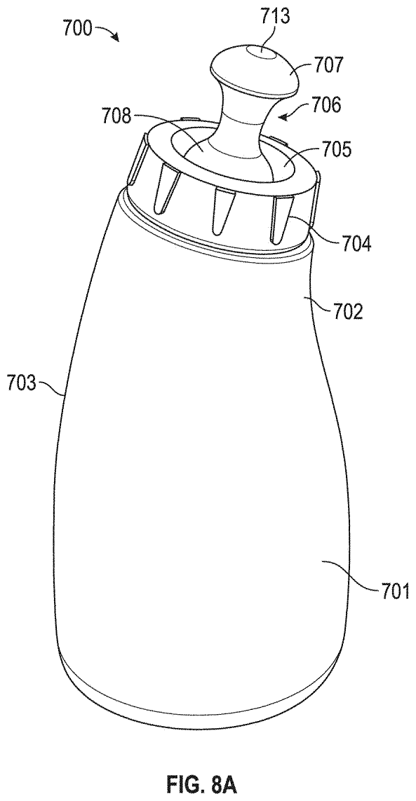

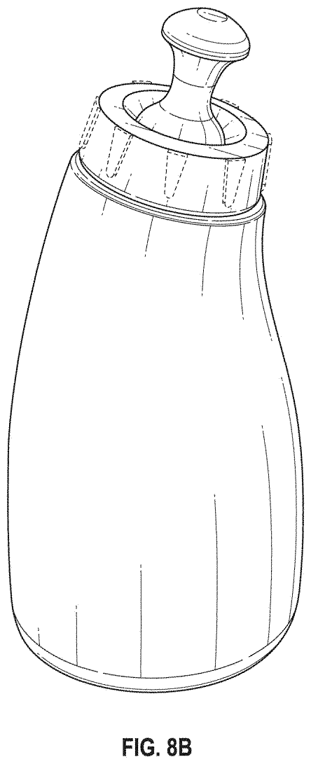

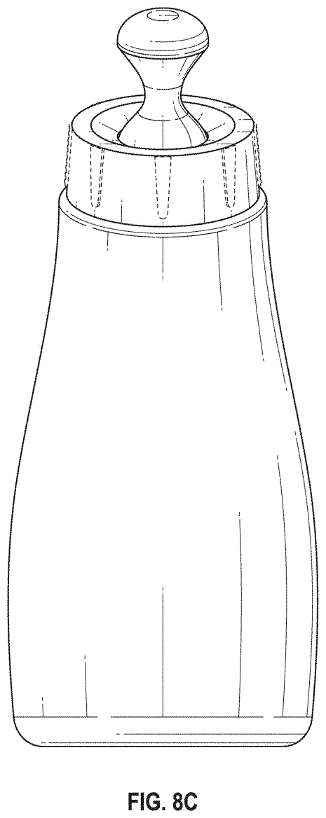

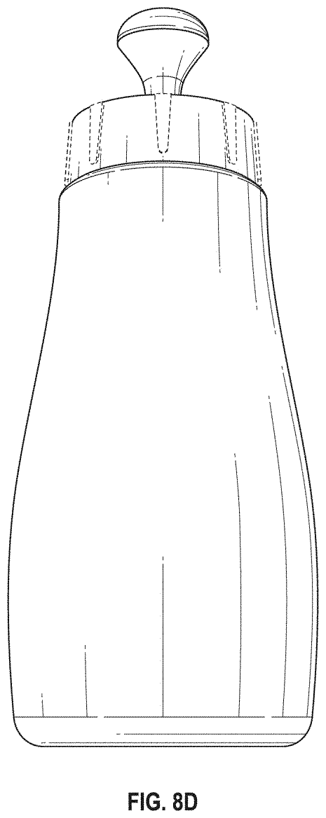

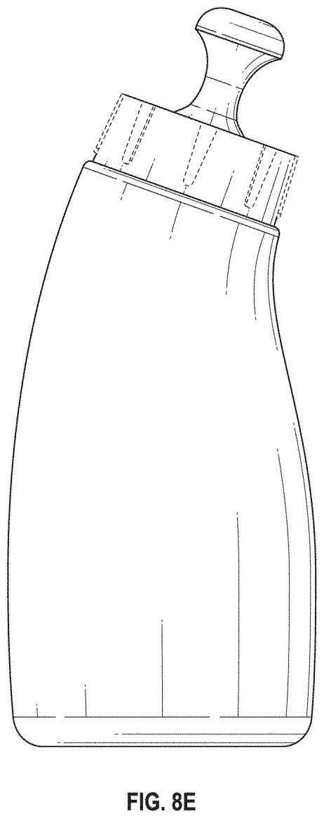

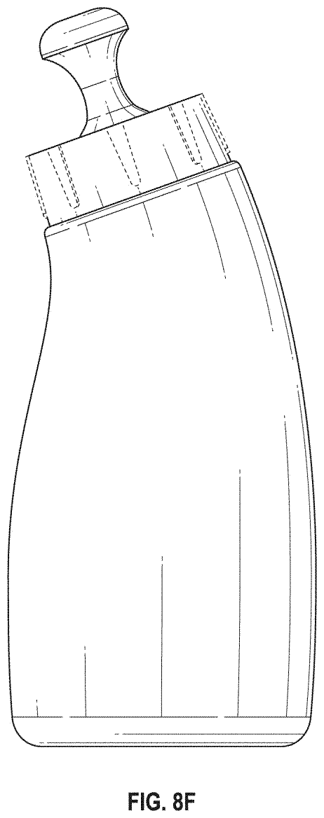

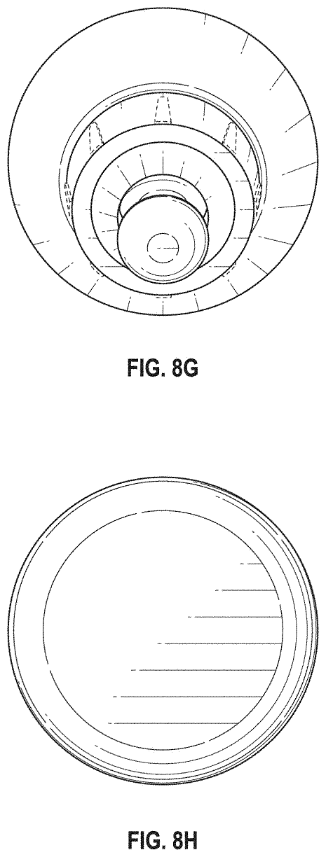

FIG. 8A shows an illustration of the nasal irrigation device 700 shown in FIG. 7 upon assembly, without outer cap 711. Visible in FIG. 8A is nasal irrigation device 700 comprising a container 701 having a flexible sidewall 703 and a neck portion 702, an inner cap 704 having a sealing ring 705, a nozzle 706 having a tip 707 defining an opening 713 and a ball joint 708. FIG. 8B is a perspective view of the nasal irrigation device shown in FIG. 8A. FIG. 8C is a front perspective view of the nasal irrigation device shown in FIG. 8A. FIG. 8D is a back perspective view of the nasal irrigation device shown in FIG. 8A. FIG. 8E is a right perspective view of the nasal irrigation device shown in FIG. 8A. FIG. 8F is a left perspective view of the nasal irrigation device shown in FIG. 8A. FIG. 8G is a top perspective view of the nasal irrigation device shown in FIG. 8A. FIG. 8H is a bottom perspective view of the nasal irrigation device shown in FIG. 8A.

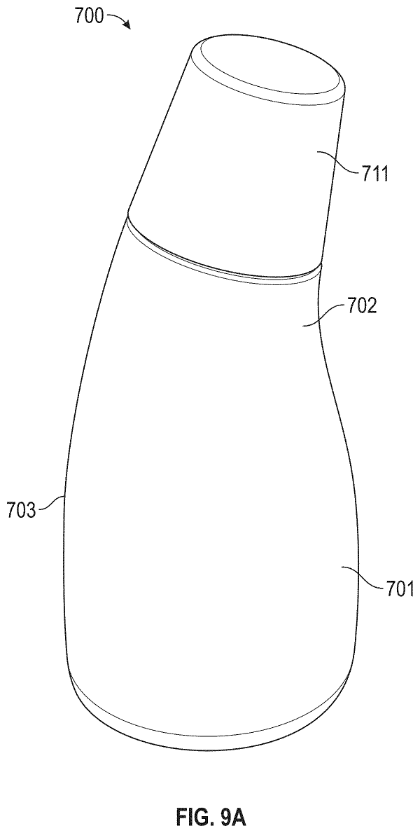

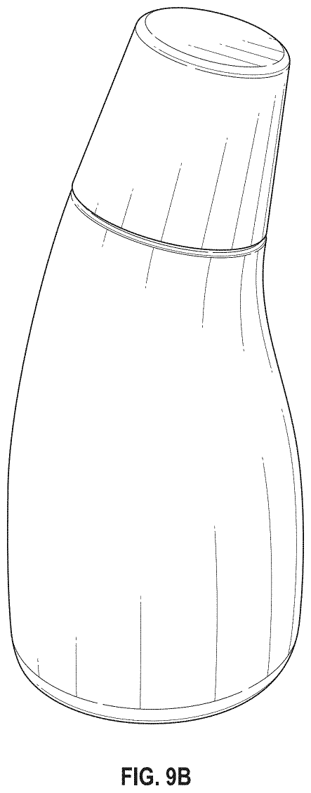

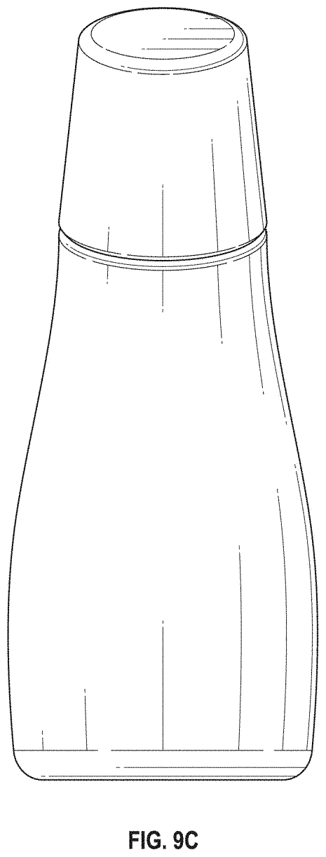

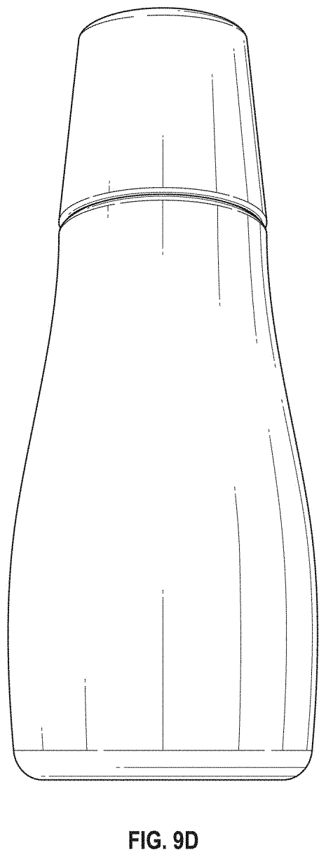

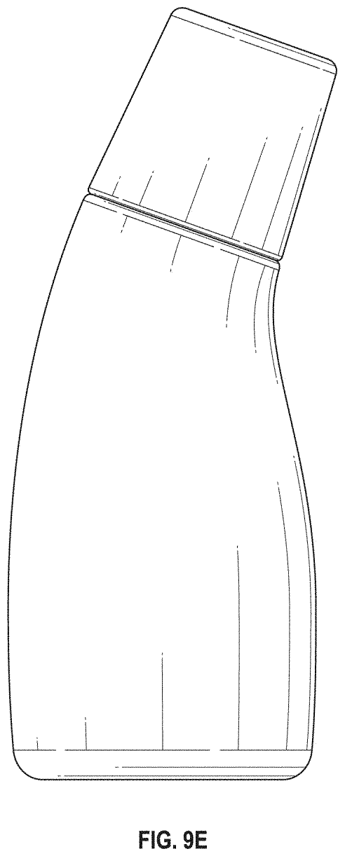

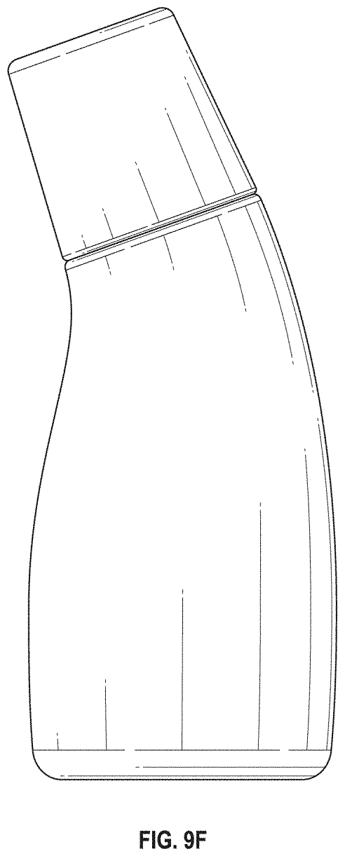

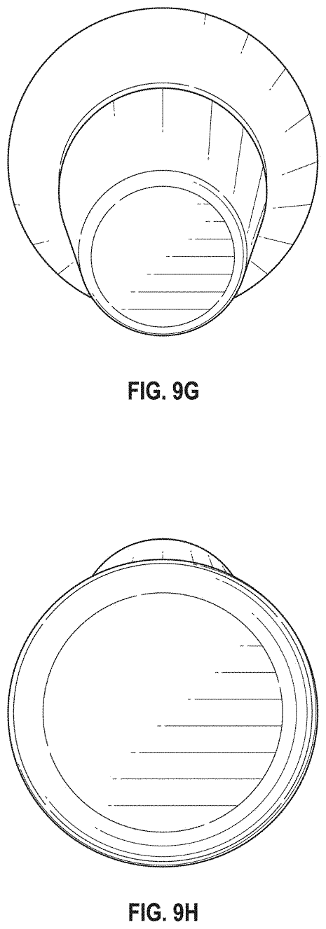

FIG. 9A shows an illustration of the nasal irrigation device 700 shown in FIG. 7 upon assembly, with outer cap 711. Visible in FIG. 9A is nasal irrigation device 700 comprising a container 701 comprising a flexible sidewall 703 and having a neck portion 702, and outer cap 711. FIG. 9B is a perspective view of the nasal irrigation device with an outer cap shown in FIG. 9A. FIG. 9C is a front perspective view of the nasal irrigation device with an outer cap shown in FIG. 9A. FIG. 9D is a back perspective view of the nasal irrigation device with an outer cap shown in FIG. 9A. FIG. 9E is a right perspective view of the nasal irrigation device with an outer cap shown in FIG. 9A. FIG. 9F is a left perspective view of the nasal irrigation device with an outer cap shown in FIG. 9A. FIG. 9G is a top perspective view of the nasal irrigation device with an outer cap shown in FIG. 9A. FIG. 9H is a bottom perspective view of the nasal irrigation device with an outer cap shown in FIG. 9A.

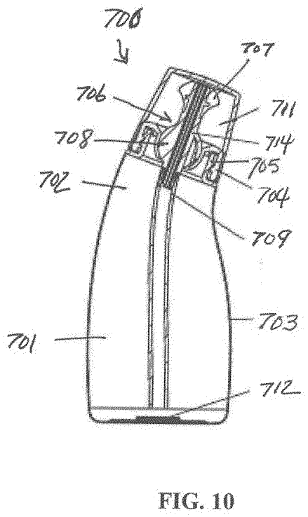

FIG. 10 shows a cross-sectional view of the nasal irrigation device 700 shown in FIG. 7 upon assembly. Visible in FIG. 10 is nasal irrigation device 700, comprising a container 701 having a flexible sidewall 703, and a neck portion 702, an inner cap 704 having a sealing ring 705, a nozzle 706 having a tip 707 and a ball joint 708, a tube 709, an outer cap 711 and a time management strip 712.

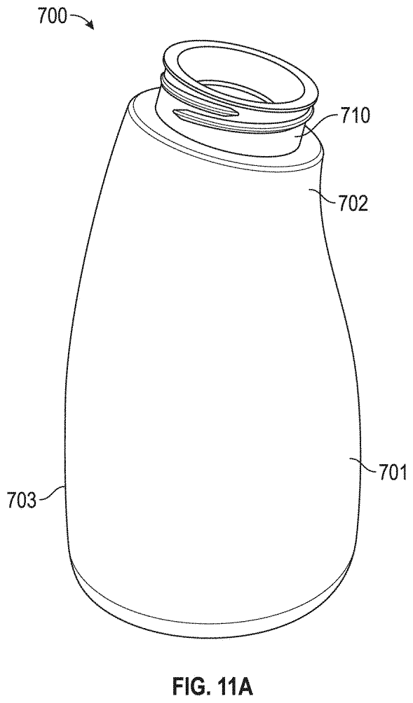

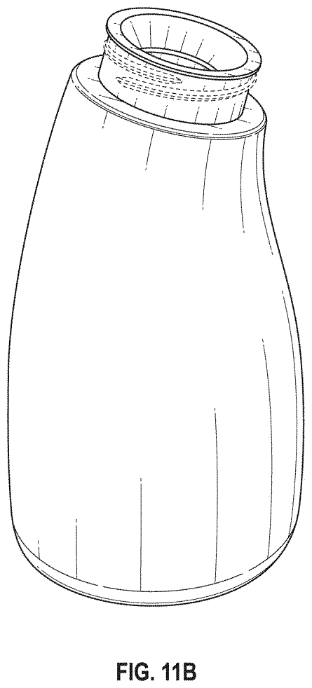

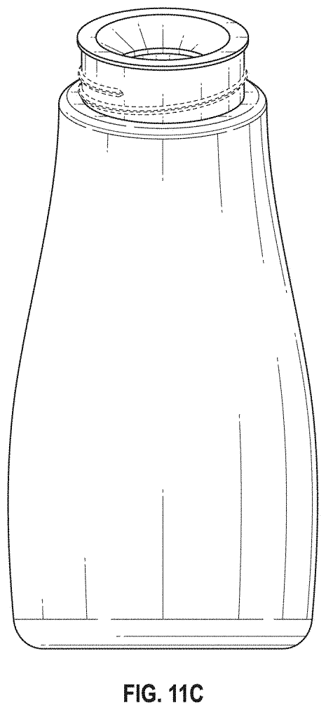

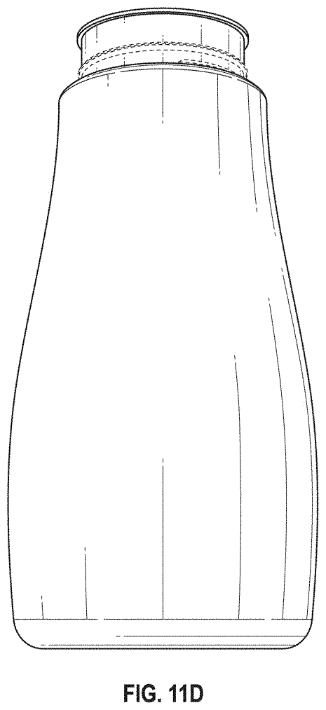

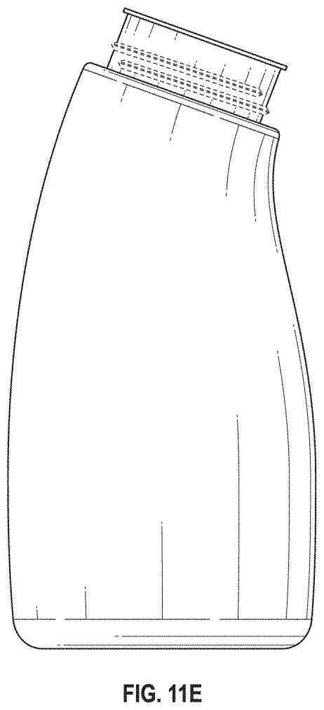

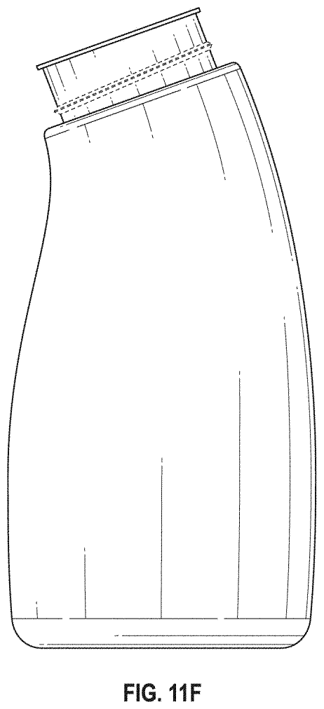

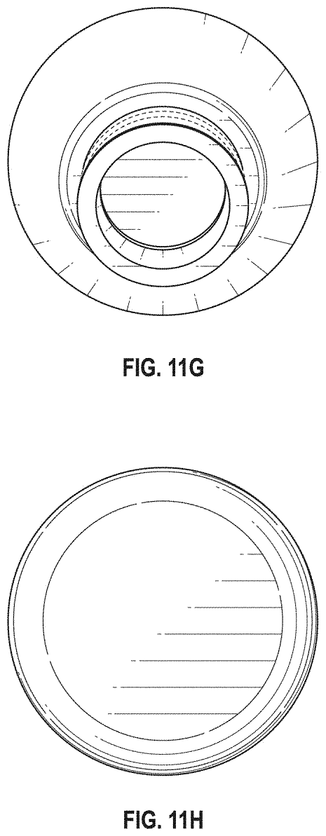

FIG. 11 shows an illustration of a container 701 of the nasal irrigation device 700 shown in FIG. 7. Container 701 comprises a flexible sidewall 703 and a neck portion 702 comprising a first fitting 710. FIG. 11B is a perspective view of the container of a nasal irrigation device shown in FIG. 11A. FIG. 11C is a front perspective view of the container of a nasal irrigation device shown in FIG. 11A. FIG. 11D is a back perspective view of the container of a nasal irrigation device shown in FIG. 11A. FIG. 11E is a right perspective view of the container of a nasal irrigation device shown in FIG. 11A. FIG. 11F is a left perspective view of the container of a nasal irrigation device shown in FIG. 11A. FIG. 11G is a top perspective view of the container of a nasal irrigation device shown in FIG. 11A. FIG. 11H is a bottom perspective view of the container of a nasal irrigation device shown in FIG. 11A.

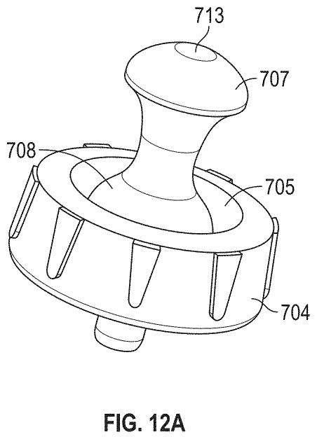

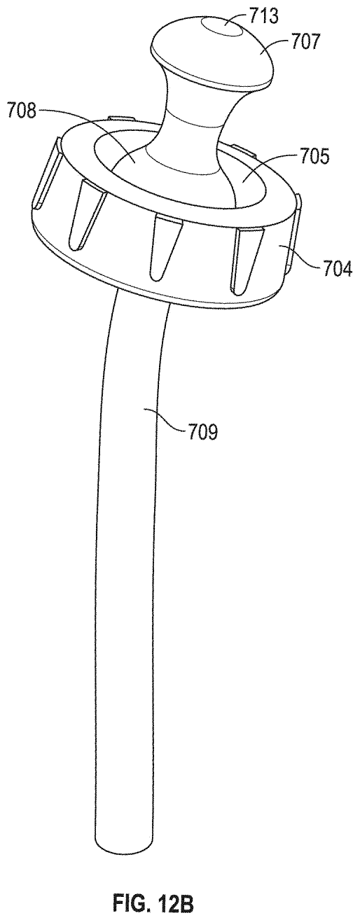

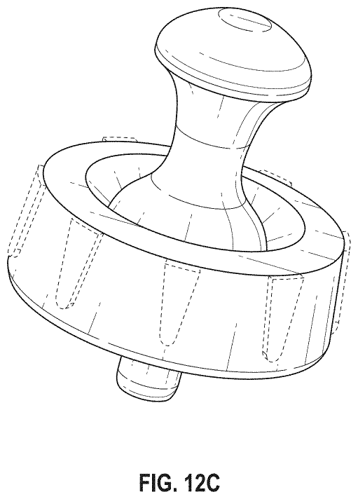

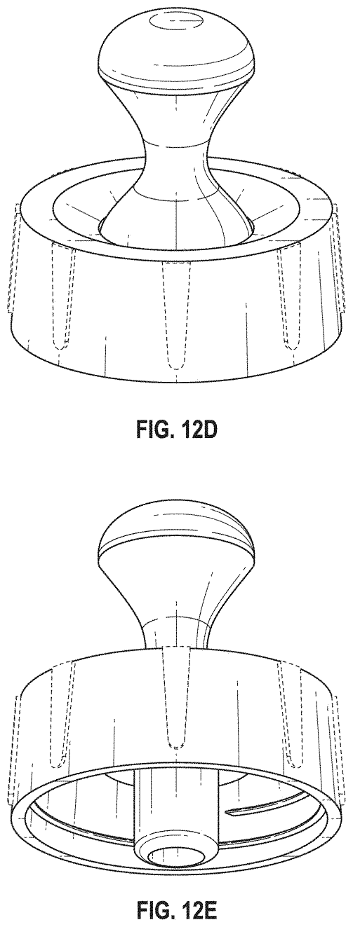

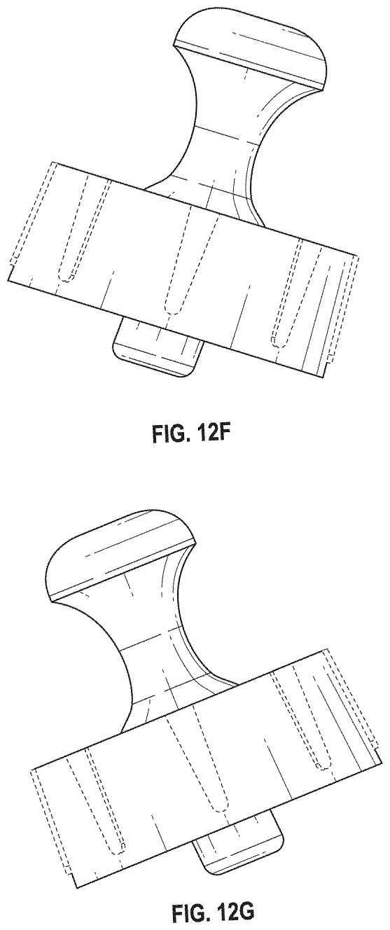

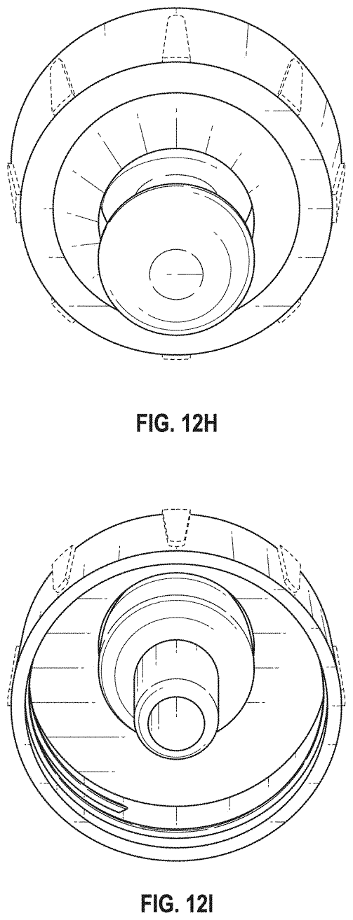

FIG. 12A shows an illustration of a nozzle and inner cap of the nasal irrigation device shown in FIG. 7. Inner cap 704 comprises sealing ring 705, the inner cap engaging the nozzle comprising a ball joint 708 and a tip 707 defining an opening 713. FIG. 12B shows an illustration of a nozzle and inner cap of the nasal irrigation device shown in FIG. 7 engaging a tube. Inner cap 704 comprises sealing ring 705, the inner cap engaging the nozzle comprising a ball joint 708 and a tip 707 defining an opening 713, the nozzle engaging tube 709. FIG. 12C is a perspective view of the nozzle and inner cap of a nasal irrigation device shown in FIG. 12A. FIG. 12D is a front perspective view of the nozzle and inner cap of a nasal irrigation device shown in FIG. 12A. FIG. 12E is a back perspective view of the nozzle and inner cap of a nasal irrigation device shown in FIG. 12A. FIG. 12F is a right perspective view of the nozzle and inner cap of a nasal irrigation device shown in FIG. 12A. FIG. 12G is a left perspective view of the nozzle and inner cap of a nasal irrigation device shown in FIG. 12A. FIG. 12H is a top perspective view of the nozzle and inner cap of a nasal irrigation device shown in FIG. 12A. FIG. 12I is a bottom perspective view of the nozzle and inner cap of a nasal irrigation device shown in FIG. 12A. FIG. 12J is a perspective view of the nozzle, inner cap and tube of a nasal irrigation device shown in FIG. 12B.

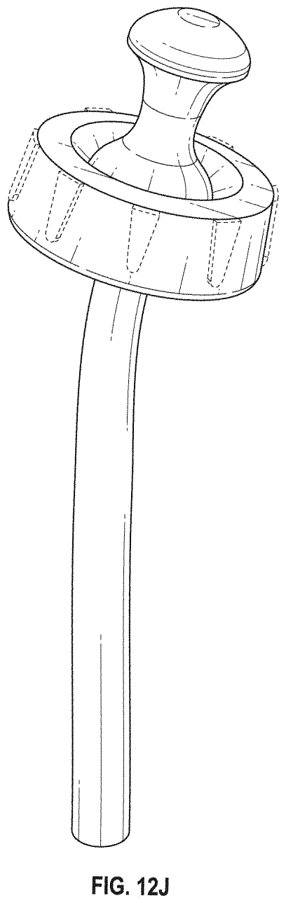

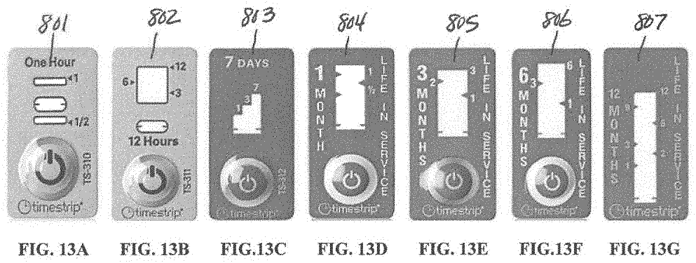

FIG. 13A shows one embodiment of a time management strip 801 for use with the present disclosure. FIG. 13B shows another embodiment of a time management strip 802 for use with the present disclosure. FIG. 13C shows another embodiment of a time management strip 803 for use with the present disclosure. FIG. 13D shows another embodiment of a time management strip 804 for use with the present disclosure. FIG. 13E shows another embodiment of a time management strip 805 for use with the present disclosure. FIG. 13F shows another embodiment of a time management strip 806 for use with the present disclosure. FIG. 13G shows another embodiment of a time management strip 807 for use with the present disclosure. Different liquid contents of the nasal irrigation device result in the device having different effective lifetimes. The different time management strips display different time periods that have elapsed since the device was first used to allow the user of the nasal irrigation device to determine how long the nasal irrigation device has been in use and when replacement is needed.

The material configuring the nasal irrigation device and component parts thereof is not limited, and may comprise any suitable material.

All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Features or functionality described with respect to certain example embodiments may be combined and sub-combined in and/or with various other example embodiments. Also, different features and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, can be at least partially performed via at least one entity or actor in any manner.

As used herein, a term “about” or “substantially” refers to a +/−10% variation from a nominal value/term. Such variation is always included in any given value/term provided herein, whether or not such variation is specifically referred thereto.

Any component described herein can include a material suitable for a medical use. The material can be, flexible, elastic, or resilient. The material can be suitable to be disinfected, sterilized, or sanitized, which can be with a hot steam, an autoclave, or others. For example, the material can include plastic, metal, rubber, shape memory, fabric, foam, or others.

The device and system of the present disclosure has been described with specific reference to certain drawings and various embodiments, but may, however, be embodied in many different forms and should not be construed as necessarily being limited to only embodiments disclosed herein. Rather, these embodiments are provided so that this disclosure is thorough and complete, and fully conveys various concepts of this disclosure to skilled artisans.

Note that various terminology used herein can imply direct or indirect, full or partial, temporary or permanent, action or inaction. For example, when an element is referred to as being “on,” “connected” or “coupled” to another element, then the element can be directly on, connected or coupled to the other element or intervening elements can be present, including indirect or direct variants. In contrast, when an element is referred to as being “directly connected” or “directly coupled” to another element, there are no intervening elements present.

Likewise, as used herein, a term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

As used in the specification and claims, the singular forms “a”, “an” and “the” include plural references unless the context clearly dictates otherwise. For example, the term “material” may include a plurality of materials unless the context clearly dictates otherwise. As used in the specification and claims, singular names or types referenced include variations within the family of said name unless the context clearly dictates otherwise. For example, a term “a” or “an” shall mean “one or more,” even though a phrase “one or more” is also used herein.

Certain terminology is used in the following description for convenience only and is not limiting. The words “lower,” “upper,” “bottom,” “top,” “front,” “back,” “left,” “right” and “sides” designate directions in the drawings to which reference is made, but are not limiting with respect to the orientation in which the various parts of the nasal irrigation device or any assembly of them may be used.

Moreover, terms “comprises,” “includes” or “comprising,” “including” when used in this specification, specify a presence of stated features, integers, steps, operations, elements, or components, but do not preclude a presence and/or addition of one or more other features, integers, steps, operations, elements, components, or groups thereof. Furthermore, when this disclosure states that something is “based on” something else, then such statement refers to a basis which may be based on one or more other things as well. In other words, unless expressly indicated otherwise, as used herein “based on” inclusively means “based at least in part on” or “based at least partially on.”

Additionally, although terms first, second, and others can be used herein to describe various elements, components, regions, layers, or sections, these elements, components, regions, layers, or sections should not necessarily be limited by such terms. Rather, these terms are used to distinguish one element, component, region, layer, or section from another element, component, region, layer, or section. As such, a first element, component, region, layer, or section discussed herein could be termed a second element, component, region, layer, or section without departing from this disclosure.

Also, unless otherwise defined, all terms (including technical and scientific terms) used herein have the same meaning as commonly understood by one of ordinary skill in an art to which this disclosure belongs. As such, terms, such as those defined in commonly used dictionaries, should be interpreted as having a meaning that is consistent with their meaning in a context of a relevant art and should not be interpreted in an idealized or overly formal sense unless expressly so defined herein.

In addition, features described with respect to certain example embodiments may be combined in or with various other example embodiments in any permutational or combinatory manner. Different features or elements of example embodiments, as disclosed herein, may be combined in a similar manner. The term “combination”, “combinatory,” or “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

Although preferred embodiments have been depicted and described in detail herein, skilled artisans know that various modifications, additions, substitutions and the like can be made without departing from spirit of this disclosure. As such, these are considered to be within the scope of the disclosure, as defined in the following claims.

1. A nasal irrigation device comprising:
a) a container comprising a top end, a bottom end, a bottom surface, an upper portion and a lower portion, the top end defining a neck portion comprising an inner surface and an outer surface;
b) an inner cap comprising an inner surface, an outer surface, a top end and a bottom end, the inner cap comprising a sealing ring proximal to the top end of the inner cap;
c) a nozzle comprising an opening, a top portion, a middle portion and a bottom portion, the nozzle defining a passageway therethrough, the top portion of the nozzle defining a tip and the middle portion of the nozzle defining a ball joint, the nozzle engaging with the sealing ring of the inner cap; and
d) a tube having a top end and a bottom end, the top end of the tube engaging with the bottom portion of the nozzle and the bottom end of the tube extending within the container from the top end of the container toward the bottom end of the container;
wherein the angle of the nozzle is adjustable relative to the container when the nasal irrigation device is assembled.

2. The nasal irrigation device of claim 1, wherein the outer surface of the neck portion comprises a first fitting and the inner surface of the inner cap comprises a second fitting, wherein the first fitting and the second fitting are designed to releasably engage with each other.

3. The nasal irrigation device of claim 1, wherein the inner surface of the neck portion comprises a third fitting and the outer surface of the inner cap comprises a fourth fitting, wherein the third fitting and the fourth fitting are designed to releasably engage with each other.

4. The nasal irrigation device of claim 1, wherein the nozzle is adjustable relative to the container when the nasal irrigation device is assembled based on a rotational position of the ball joint relative to the sealing ring of the inner cap.

5. The nasal irrigation device le of claim 1, wherein the opening has a stellate shape.

6. The nasal irrigation device of claim 1, further comprising an outer cap comprising a top end, a bottom end, an outer surface and an inner surface, the inner surface proximal to the bottom end of said outer cap designed to engage with the outer surface of the inner cap.

7. The nasal irrigation device of claim 1, further comprising a time management strip attached to the bottom surface of the container.

8. The nasal irrigation device of claim 1, wherein the container is comprised of a flexible polymer.

9. The nasal irrigation device of claim 8, wherein the polymer comprises zinc or silver.

10. The nasal irrigation device of claim 8, wherein the polymer comprises low-density polyethylene (LDPE)

11. The nasal irrigation device of claim 1, wherein the tip of the nozzle is designed to fit within a nostril of a patient.

12. The nasal irrigation device of claim 1, wherein the tip of the nozzle has a semicircular shape.

13. The nasal irrigation device of claim 1, wherein the tip is comprised of rubber or a thermoplastic elastomer.

14. The nasal irrigation device of claim 1, wherein the tip is comprised of a material that reduces the risk of at least one of nasal bleeding or septum perforation when the tip is inserted into a nostril.

15. The nasal irrigation device of claim 1, wherein the tip is in the shape of a reverse taper.

16. The nasal irrigation device of claim 15, wherein the reverse taper shape provides for improved nasal fitting at the nostril level.

17. The nasal irrigation device of claim 1, wherein the middle portion of the nozzle is comprised of plastic.

18. The nasal irrigation device of claim 1, wherein the middle portion of the nozzle comprises acrylonitrile butadiene styrene (ABS).

19. The nasal irrigation device of claim 1, wherein the container further comprises a liquid, and wherein the nozzle is capable of varying the amount of the liquid passing through the nozzle.

20. The nasal irrigation device of claim 19, wherein the liquid is a saline solution.

21. The nasal irrigation device of claim 1, wherein the container further comprises a flexible sidewall.

22. The nasal irrigation device of claim 21, wherein the container further comprises a liquid, and wherein the nozzle is configured to create a turbulent stream of liquid exiting the nozzle when the sidewall is squeezed.

23. The nasal irrigation device of claim 21, wherein the container further comprises a liquid, and wherein the nozzle is configured to create a level of turbulence in a stream of liquid exiting the nozzle when the sidewall is squeezed, such that the turbulent stream of liquid is directed toward and sufficient to enter and flush at least a substantial portion of a sinus passage.

24. The nasal irrigation device of claim 1, wherein the tip of the nozzle defines a stellate shaped opening.

25. The nasal irrigation device of claim 1, wherein the tip of the nozzle defines an opening that increases surface area dispersion and intranasal turbulence.

26. The nasal irrigation device of claim 25, wherein the surface area dispersion and intranasal turbulence are sufficient to generate a flooding mechanism for irrigation of a sinus cavity.

27. The nasal irrigation device of claim 1, wherein the upper portion of the container is angled relative to the lower portion of the container.

28. The nasal irrigation device of claim 1, wherein the inner cap comprises ABS.

29. The nasal irrigation device of claim 1, wherein the sealing ring comprises a thermoplastic elastomer.

30. The nasal irrigation device of claim 2, wherein the first fitting and the second fitting are threadably engaged when the nasal irrigation device is assembled.

31. The nasal irrigation device of claim 2, wherein the first fitting and the second fitting form a snap fit connection when the nasal irrigation device is assembled.

32. The nasal irrigation device of claim 3, wherein the third fitting and the fourth fitting are threadably engaged when the nasal irrigation device is assembled.

33. The nasal irrigation device of claim 3, wherein the third fitting and the fourth fitting form a snap fit connection when the nasal irrigation device is assembled.

34. The nasal irrigation device of claim 1, wherein upon engagement of the ball joint with the sealing ring the nozzle comprises multidirectional positioning capabilities.

35. The nasal irrigation device of claim 34, wherein the nozzle is positioned to irrigate in at least one of a superior or inferior arrangement.
